(19) United States
(12) Patent Application Publication
(10) Pub. No.: US 2009/0320354 A1
(43) Pub. Date: Dec. 31, 2009

(54) BRANCHED DECYL NITRATES AND THEIR USE AS COMBUSTION IMPROVERS AND/OR CETANE NUMBER IMPROVERS IN FUELS

(75) Inventors: Claudius Kormann, Schifferstadt (DE); Uwe Rebholz, Mehlingen (DE); Gero Nordmann, Heidelberg (DE); Jörn Karl, Beijing (CN); Lothar Karrer, Pfungstadt (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/524,169

(22) PCT Filed: Jan. 25, 2008
(86) PCT No.: PCT/EP2008/050883

§ 371 Date: Jul. 23, 2009

(30) Foreign Application Priority Data
Jan. 29, 2007 (EP) 07101307.2

(51) Int. Cl. C10L 1/232; C07C 203/04; C10L 1/23

(52) U.S. Cl. 44/325; 558/480; 44/324

(57) ABSTRACT

Branched decyl nitrates of the formula R¹R²CH—CH₂—O—NO₂ in which R¹ is an n-propyl or isopropyl radical and R² is a linear or branched alkyl radical having 5 carbon atoms are suitable as combustion improvers and/or cetane number improvers in fuels.

The present invention relates to specific branched decyl nitrates and to a mixture thereof, to the preparation of these branched decyl nitrates and to this mixture, and also to a fuel and to a fuel additive concentrate which comprise these branched decyl nitrates or this mixture, and to the use of these branched decyl nitrates or of this mixture as combustion improvers and/or cetane number improvers in fuels.

Organic nitrates have been known for some time as ignition accelerants in fuels, especially in middle distillates. Organic nitrates have also been used for some time to increase the cetane number in diesel fuels. Higher cetane numbers lead to more rapid engine starts, especially in cold weather, to lower engine noise, to more complete combustion, to less evolution of smoke and, under some circumstances, to less injector carbonization.

Typical organic nitrates which are suitable as combustion improvers in gasoline fuels or as cetane number improvers in diesel fuels are nitrates of short- and medium-chain linear and branched alkanols and nitrates of cycloalkanols, such as n-hexyl nitrate, 2-ethylhexyl nitrate, n-heptyl nitrate, n-octyl nitrate, isooctyl nitrate, sec-octyl nitrate, n-nonyl nitrate, n-decyl nitrate, n-dodecyl nitrate, cyclopentyl nitrate, cyclohexyl nitrate, methylcyclohexyl nitrate and isopropylcyclohexyl nitrate. The most commercially significant thereof is 2-ethylhexyl nitrate. These organic nitrates and their use as combustion improvers or cetane number improvers are described, for example, in the documents U.S. Pat. No. 6,676,715 B2, U.S. Pat. No. 4,473,378, US 2003/0110684 A1, U.S. Pat. No. 7,018,433 B2, U.S. Pat. No. 5,782,937 and U.S. Pat. No. 7,029,506 B2.

U.S. Pat. No. 4,479,905 describes the preparation of organic nitrates by nitrating a mixture of aliphatic primary alcohols, for example n-octanol, 2-ethylhexanol, n-decanol or 2-ethyloctanol, and alkoxyalkanols, for example 2-ethoxyethanol or 2-butoxyethanol, by means of nitric acid and sulfuric acid. The nitrate mixtures thus prepared are suitable as cetane number improvers in diesel fuels.

The organic nitrates described in the prior art as combustion improvers and/or cetane number improvers have a series of disadvantages, especially lack of thermal stability, excessively high volatility and insufficient efficacy. An excessively high volatility as a consequence of a relatively low boiling point, which occurs naturally in the case of compounds of relatively low molecular weight, has an adverse effect on odor, on flashpoint and hence on the safety risk associated with the handling of such compounds. The experience of the person skilled in the relevant art teaches that, although the volatility is expected to fall with higher molecular weights, the efficacy as a combustion improver and/or cetane number improver declines.

The solutions proposed in the prior art for alleviating the thermal stability problems, such as the mixing of highly effective but particularly explosion-sensitive organic nitrates such as C₁-C₃-alkyl nitrates with higher molecular weight but less effective nitrates such as octyl nitrates (described in U.S. Pat. No. 4,473,378), or such as the stabilization of cetane improvers with heterocyclic compounds having relatively long-chain hydrocarbon groups (described in U.S. Pat. No. 6,676,715 B2), are not convincing either.

It was therefore an object of the invention to provide organic nitrates which have a better effectiveness than or at least the same effectiveness as combustion improvers or cetane number improvers as the market standard 2-ethylhexyl nitrate, but additionally a lower volatility, a higher flashpoint and a higher temperature of autocatalytic decomposition, i.e. a better thermal stability, than it. Moreover, a reduction in the percentage nitrogen content of the organic nitrates to be prepared is also desirable in order to minimize the content of nitrogen oxides in the exhaust gases. Not least, good low-temperature performance is also desirable.

It has been found that, surprisingly, specific branched decyl nitrates achieve this object.

The nitrate formed from 2-ethyloctanol as the branched decanol is already known from U.S. Pat. No. 4,479,905. Since the specific branched decyl nitrates discovered, however, constitute novel substances, the present invention provides branched decyl nitrates of the formula I

R¹R²CH—CH₂—O—NO₂ (I)

in which R¹ is an n-propyl or isopropyl radical and R² is a linear or branched alkyl radical having 5 carbon atoms.

The alkyl radical having 5 carbon atoms for R² may, for example, be n-pentyl, 1-methyl-butyl, 2-methylbutyl, 3-methylbutyl, 1-ethylpropyl, 1,1-dimethylpropyl, 2,2-dimethyl-propyl or 1,2-dimethylpropyl.

Typical branched decyl nitrates of the formula I are:

(Ia) 2-n-propylheptyl nitrate (R¹=n-propyl, R²=n-pentyl)
(Ib) 2-isopropylheptyl nitrate (R¹=isopropyl, R²=n-pentyl)
(Ic) 2-n-propyl-4-methylhexyl nitrate (R¹=n-propyl, R²=2-methylbutyl)
(Id) 2-isopropyl-4-methylhexyl nitrate (R¹=isopropyl, R²=2-methylbutyl)
(Ie) 2-n-propyl-5-methylhexyl nitrate (R¹=n-propyl, R²=3-methylbutyl)
(If) 2-isopropyl-5-methylhexyl nitrate (R¹=isopropyl, R²=3-methylbutyl)
(Ig) 2-n-propyl-4,4-dimethylpentyl nitrate (R¹=n-propyl, R²=2,2-dimethylpropyl)
(Ih) 2-isopropyl-4,4-dimethylpentyl nitrate (R¹=isopropyl, R²=2,2-dimethylpropyl)

The branched decyl nitrates of the formula I mentioned are prepared appropriately by nitrating the corresponding branched decanols by means of a mixture of nitric acid and sulfuric acid (customarily referred to as “nitrating acid”). Preference is given to working with a mixture of approx. 65% by weight nitric acid and approx. 96% by weight sulfuric acid in a volume ratio of approx. 2:3 as the nitrating acid. The nitrating acid is customarily used in a high excess compared to the branched decanol to be nitrated, for example in a molar HNO₃ to decanol ratio of from 10:1 to 25:1. The nitration reaction is performed generally at temperatures of from −10 to +25° C., preferably at from 0 to +15° C., with sufficient cooling. Owing to the strong exothermicity of the nitrating reaction, suitable safety measures should be observed here. Such a preparation process is described in U.S. Pat. No. 4,479,905 for other alkyl nitrates.

An example of a branched decanol to be used for the above-described nitration is 2-n-propylheptanol. A possible synthetic route for 2-n-propylheptanol is the condensation of 1-pentanol or of a mixture of methylbutan-1-ols in the presence of alkali metal hydroxide, e.g. potassium hydroxide, at elevated temperatures.

Of particular interest for the present invention is a specific mixture of the branched decyl nitrates mentioned, which constitutes a novel substance mixture. The present invention therefore also provides a mixture of branched decyl nitrates which comprises, as components,

from 10 to 99% by weight, especially from 40 to 97% by weight, of 2-n-propylheptyl nitrate (compound Ia),
from 1 to 90% by weight, especially from 3 to 60% by weight, of 2-n-propyl-4-methyl-hexyl nitrate and/or 2-n-propyl-5-methylhexyl nitrate (compound Ic or Ie) and
from 0 to 50% by weight, especially from 0 to 30% by weight, of other branched decyl nitrates, especially those of the formula I, for example one or more of compounds Ib, Id, Ie, If, Ig or Ih,
where the sum of the components mentioned adds up to 100% by weight. The proportions of the individual branched decyl nitrates in such a mixture can be determined easily with customary analytical methods such as gas chromatography.

A typical representative of this mixture is technical-grade 2-n-propylheptyl nitrate, which generally comprises from 80 to 95% by weight of the pure compound Ia, from 5 to 15% by weight of the pure compound(s) Ic and/or Ie and from 0 to 10% by weight of other branched decyl nitrates, where the sum of all branched decyl nitrates adds up to 100% by weight. A typical representative of the other branched decyl nitrates is 2-isopropylheptyl nitrate (compound Ib), which is readily detectable even in small amounts with the customary analytical methods. In addition, this mixture, as a result of the preparation, may also comprise by-products in small amounts. Technical 2-n-propylheptanol and its preparation as a precursor for this technical-grade 2-n-propylheptyl nitrate are described, for example, in U.S. Pat. No. 4,426,542.

The mixture of branched decyl nitrates mentioned, especially the above-described technical 2-n-propylheptyl nitrate, is prepared appropriately by a synthesis sequence which is characterized by the following process steps:
(a) hydroformylating butenes to C₅-aldehydes,
(b) dimerizing the C₅-aldehydes by aldol condensation to give the corresponding C₁₀-acrolein derivates,
(c) hydrogenating the C₁₀-acrolein derivates to the corresponding decanols and
(d) nitrating the decanols by means of a mixture of nitric acid and sulfuric acid to give the mixture of branched decyl nitrates.

Process step (a) is described, for example, in U.S. Pat. No. 4,287,370. Hydroformylation of a C₄ hydrocarbon stream which, as the main component, comprises 1-butene as well as 2-butene, isobutene and if appropriate butanes by means of a rhodium catalyst complex affords, through this hydroformylation process used with preference, a mixture of n-valeraldehyde (n-pentanal), isovaleraldehyde (2-methylbutanal) and if appropriate secondary components such as 3-methylbutanal. The weight ratio of n-valeraldehyde to isovaleraldehyde varies according to the feedstock composition and reaction conditions, and is usually in the range from 8:1 to 20:1.

Process step (b) is described, for example, in U.S. Pat. No. 5,434,313. Aldol condensation, preferably by means of aqueous alkali metal hydroxide, affords, through this dimerization process used with preference, from the n-valeraldehyde obtained in process step (a), 2-n-propyl-3-n-butylacrolein (2-n-propyl-2-heptenal) and, from the isovaleraldehyde obtained, 2-n-propyl-3-sec-butylacrolein (2-n-propyl-4-methyl-2-hexenal). Any isomeric C₅ aldehydes also present in small amounts may form further isomeric C₁₀-acrolein derivates.

In process step (c), the C₁₀-acrolein derivatives obtained in process step (b) are hydrogenated by customary methods with hydrogen to give the corresponding branched decanols. To this end, typically heterogeneous hydrogenation catalysts based, for example, on nickel, cobalt or copper are used, for example Raney nickel or cobalt on kieselguhr. This forms 2-n-propylheptanol from the 2-n-propyl-3-n-butylacrolein (2-n-propyl-2-heptenal), and 2-n-propyl-4-methylhexanol from the 2-n-propyl-3-sec-butylacrolein (2-n-propyl-4-methyl-2-hexenal).

The branched decanols obtained in process step (c) are nitrated in process step (d) by means of a mixture of nitric acid and sulfuric acid (typically referred to as “nitrating acid”). Preference is given to working with a mixture of approx. 65% by weight nitric acid and approx. 96% by weight sulfuric acid in a volume ratio of approx. 2:3 as the nitrating acid. The nitrating acid is used typically in a high excess compared to the branched decanols to be nitrated, for example in a molar HNO₃ to decanols ratio of from 10:1 to 25:1. The nitration reaction is performed generally at temperatures of from −10 to +25° C., preferably at from 0 to +15° C., with sufficient cooling. Owing to the strong exothermicity of the nitration reaction, suitable safety measures should be observed here. Such a preparation process is described for other alkyl nitrates in U.S. Pat. No. 4,479,905.

Since the branched decyl nitrates mentioned and especially the mixture of branched decyl nitrates mentioned exhibit the desired effect in fuels as combustion improvers or cetane number improvers, the present invention further provides an additized fuel which comprises a major proportion of a base fuel, a minor proportion of at least one of the branched decyl nitrates mentioned or of the mixture of branched decyl nitrates mentioned, and also, if appropriate, further customary fuel additives in the amounts customary therefor.

The inventive additized fuel comprises generally from 20 to 2000 ppm by weight, especially from 100 to 1500 ppm by weight, in particular from 250 to 1200 ppm by weight of branched decyl nitrates. Typical dosage rates are 500 ppm by weight or 1000 ppm by weight.

The inventive branched decyl nitrates may in principle be used to improve the combustion in all base fuel types, for example in gasoline fuel, in middle distillate fuel, here especially in diesel fuel and heating oil, in turbine fuel or in kerosene.

In a preferred embodiment, the inventive additized fuel comprises a gasoline fuel as the base fuel. Useful gasoline fuels in this context are all commercial gasoline fuel compositions. As a typical representative, Eurosuper base fuel according to EN 228, which is customary on the market, shall be mentioned here. Gasoline fuel compositions of the specification according to WO 00/47698 are also possible fields of use for the present invention.

In a further preferred embodiment, the inventive additized fuel comprises a middle distillate fuel, especially a diesel fuel, as the base fuel. The middle distillate fuels and diesel fuels are typically crude oil raffinates which generally have a boiling range of from 100° C. to 400° C. These are usually distillates having a 95% point up to 360° C. or even higher. They may also be so-called “ultra low sulfur diesel” or “city diesel”, characterized by a maximum 95% point of, for example, 345° C. and a maximum sulfur content of 0.005% by weight, or by a 95% point of, for example, 285° C. and a maximum sulfur content of 0.001% by weight. In addition to the diesel fuels obtainable by refining, whose main constituents are relatively long-chain paraffins, those which are obtainable by coal gasification or gas liquefaction [“gas to liquid” (GTL) fuels] are suitable. Also suitable are mixtures of the aforementioned diesel fuels with renewable fuels such as biodiesel or bioethanol. At the present time, diesel fuels with a low sulfur content are of particular interest, i.e. with a sulfur content of less than 0.05% by weight, preferably of less than 0.02% by weight, in particular of less than 0.005% by weight and especially of less than 0.001% by weight of sulfur. Diesel fuels may also comprise water, for example in an amount up to 20% by weight, for example in the form of diesel-water microemulsions or as so-called “white diesel”.

In addition, the inventive additized fuel may also comprise a heating oil. Heating oils are, for example, low-sulfur or sulfur-rich mineral oil raffinates or bituminous or brown coal distillates which typically have a boiling range of from 150 to 400° C. Heating oils may be standard heating oil according to DIN 51603-1, which has a sulfur content of from 0.005 to 0.2% by weight, or are low-sulfur heating oils having a sulfur content of from 0 to 0.005% by weight. Examples of heating oil include in particular heating oil for domestic oil-fired boilers or EL heating oil.

In addition, the inventive additized fuel may also comprise a turbine fuel. This is, for example, a liquid turbine fuel customary in civil or military aviation. These include, for example, fuels of the designation Jet Fuel A, Jet Fuel A-1, Jet Fuel B, Jet Fuel JP-4, JP-5, JP-7, JP-8 and JP-8+100. Jet A and Jet A-1 are commercially available turbine fuel specifications based on kerosene. Jet B is a more widely cut fuel based on naphtha and kerosene fractions. JP-4 is equivalent to Jet B. JP-5, JP-7, JP-8 and JP-8+100 are military turbine fuels, as used, for example, by the marines and air force. Some of these standards relate to formulations which already comprise further additives such as corrosion inhibitors, icing inhibitors, static dissipators, etc.

The inventive branched decyl nitrates or the inventive mixture of branched decyl nitrates may be added to the base fuel, especially to the diesel fuel, either alone or in the form of diesel performance packages. Such diesel performance packages constitute fuel additive concentrates and generally comprise, as well as solvents, a series of further components as coadditives, for example carrier oils, cold flow improvers, corrosion inhibitors, demulsifiers, dehazers, antifoams, further cetane number improvers, further combustion improvers, antioxidants or stabilizers, antistats, metallocenes, metal deactivators, solubilizers, markers and/or dyes.

In a preferred embodiment, the inventive additized fuel, here especially gasoline fuels and diesel fuels, as well as the inventive branched decyl nitrates or the inventive mixture of branched decyl nitrates, which are referred to hereinafter as component (A), comprises, as further fuel additives, at least one detergent, referred to hereinafter as component (B).

Detergents or detergent additives (B) refer typically to deposition inhibitors for fuels. The detergents are preferably amphiphilic substances which have at least one hydrophobic hydrocarbon radical having a number-average molecular weight 0 of from 85 to 20 000, especially from 300 to 5000, in particular from 500 to 2500, and have at least one polar moiety which is selected from
(Ba) mono- or polyamino groups having up to 6 nitrogen atoms, at least one nitrogen atom having basic properties;
(Bb) nitro groups, if appropriate in combination with hydroxyl groups;
(Bc) hydroxyl groups in combination with mono- or polyamino groups, at least one nitrogen atom having basic properties;
(Bd) carboxyl groups or their alkali metal or alkaline earth metal salts;
(Be) sulfonic acid groups or their alkali metal or alkaline earth metal salts;
(Bf) polyoxy-C₂-C₄-alkylene moieties which are terminated by hydroxyl groups, mono- or polyamino groups, at least one nitrogen atom having basic properties, or by carbamate groups;
(Bg) carboxylic ester groups;
(Bh) moieties which derive from succinic anhydride and have hydroxyl and/or amino and/or amido and/or imido groups; and/or
(Bi) moieties obtained by Mannich reaction of substituted phenols with aldehydes and mono- or polyamines.

The hydrophobic hydrocarbon radical in the above detergent additives, which ensures the adequate solubility in the fuel oil composition, has a number-average molecular weight (Mₙ) of from 85 to 20 000, especially from 300 to 5000, in particular from 500 to 2500. Typical hydrophobic hydrocarbon radicals, especially in conjunction with the polar moieties (Ba), (Bc), (Bh) and (Bi), include relatively long-chain alkyl or alkenyl groups, especially the polypropenyl, polybutenyl and polyisobutenyl radical, each having Mₙ=from 300 to 5000, especially from 500 to 2500, in particular from 700 to 2300.

Examples of the above groups of detergent additives include the following:

Additives comprising mono- or polyamino groups (Ba) are preferably polyalkenemono- or polyalkenepolyamines based on polypropene or conventional (i.e. having predominantly internal double bonds) polybutene or polyisobutene having Mₙ=from 300 to 5000. When polybutene or polyisobutene having predominantly internal double bonds (usually in the β- and γ-position) is used as starting material in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be, for example, ammonia, monoamines or polyamines, such as dimethylaminopropylamine, ethylenediamine, diethylenetriamine, triethylenetetramine or tetraethylenepentamine. Corresponding additives based on polypropene are described in particular in WO-A-94/24231.

Further preferred additives comprising monoamino groups (Ba) are the hydrogenation products of the reaction products of polyisobutenes having an average degree of polymerization P of from 5 to 100 with nitrogen oxides or mixtures of nitrogen oxides and oxygen, as described in particular in WO-A-97/03946.

Further preferred additives comprising monoamino groups (Ba) are the compounds obtainable from polyisobutene epoxides by reaction with amines and subsequent dehydration and reduction of the amino alcohols, as described in particular in DE-A-196 20 262.

Additives comprising nitro groups (Bb), if appropriate in combination with hydroxyl groups, are preferably reaction products of polyisobutenes having an average degree of polymerization P=from 5 to 100 or from 10 to 100 with nitrogen oxides or mixtures of nitrogen oxides and oxygen, as described in particular in WO-A-96/03367 and WO-A-96/03479. These reaction products are generally mixtures of pure nitropolyisobutenes (e.g. α,β-dinitropolyisobutene) and mixed hydroxynitropolyiso-butenes (e.g. o-nitro-β-hydroxypolyisobutene).

Additives comprising hydroxyl groups in combination with mono- or polyamino groups (Bc) are in particular reaction products of polyisobutene epoxides obtainable from polyisobutene having preferably predominantly terminal double bonds and Mₙ=from 300 to 5000, with ammonia or mono- or polyamines, as described in particular in EP-A 476 485.

Additives comprising carboxyl groups or their alkali metal or alkaline earth metal salts (Bd) are preferably copolymers of C₂-C₄₀-olefins with maleic anhydride which have a total molar mass of from 500 to 20 000 and of whose carboxyl groups some or all have been converted to the alkali metal or alkaline earth metal salts and any remainder of the carboxyl groups has been reacted with alcohols or amines. Such additives are disclosed in particular by EP-A-307 815. Such additives serve mainly to prevent valve seat wear and can, as described in WO-A-87/01126, advantageously be used in combination with customary fuel detergents such as poly(iso)buteneamines or polyetheramines.

Additives comprising sulfonic acid groups or their alkali metal or alkaline earth metal salts (Be) are preferably alkali metal or alkaline earth metal salts of an alkyl sulfosuccinate, as described in particular in EP-A-639 632. Such additives serve mainly to prevent valve seat wear and can be used advantageously in combination with customary fuel detergents such as poly(iso)buteneamines or polyetheramines.

Additives comprising polyoxy-C₂-C₄-alkylene moieties (Bf) are preferably polyethers or polyether amines which are obtainable by reaction of C₂-C₆₀-alkanols, C₆-C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl group or amino group and, in the case of the polyether amines, by subsequent reductive amination with ammonia, monoamines or polyamines. Such products are described in particular in EP-A-310 875, EP-A-356 725, EP-A-700 985 and U.S. Pat. No. 4,877,416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (Bg) are preferably esters of mono-, di- or tricarboxylic acids with long-chain alkanols or polyols, in particular those having a minimum viscosity of 2 mm²/s at 100° C., as described in particular in DE-A-38 38 918. The mono-, di- or tricarboxylic acids used may be aliphatic or aromatic acids, and particularly suitable ester alcohols or ester polyols are long-chain representatives having, for example, from 6 to 24 carbon atoms. Typical representatives of the esters are adipates, phthalates, isophthalates, terephthalates and trimellitates of isooctanol, of isononanol, of isodecanol and of isotridecanol. Such products also have carrier oil properties.

Additives comprising moieties derived from succinic anhydride and having hydroxyl and/or amino and/or amido and/or imido groups (Bh) are preferably corresponding derivatives of alkyl- or alkenyl-substituted succinic anhydride and especially the corresponding derivatives of polyisobutenylsuccinic anhydride which are obtainable by reacting conventional or highly reactive polyisobutene having Mₙ=from 300 to 5000 with maleic anhydride by a thermal route or via the chlorinated polyisobutene. Particular interest attaches to derivatives with aliphatic polyamines such as ethylenediamine, diethylenetriamine, triethylenetetramine or tetraethylenepentamine. The moieties having hydroxyl and/or amino and/or amido and/or imido groups are, for example, carboxylic acid groups, acid amides of monoamines, acid amides of di- or polyamines which, in addition to the amide function, also have free amine groups, succinic acid derivatives having an acid and an amide function, carboximides with monoamines, carboximides with di- or polyamines which, in addition to the imide function, also have free amine groups, or diimides which are formed by the reaction of di- or polyamines with two succinic acid derivatives. Such fuel additives are described in particular in U.S. Pat. No. 4,849,572.

Additives comprising moieties (Bi) obtained by Mannich reaction of substituted phenols with aldehydes and mono- or polyamines are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or highly reactive polyisobutene having Mₙ=from 300 to 5000. Such “polyisobutene-Mannich bases” are described in particular in EP-A-831 141.

For a more precise definition of the fuel additives detailed individually, reference is explicitly made here to the disclosures of the abovementioned prior art documents.

Particular preference is given to detergent additives from group (Bh). These are preferably the reaction products of alkyl- or alkenyl-substituted succinic anhydrides, especially of polyisobutenylsuccinic anhydrides, with amines and/or alcohols. These are thus derivatives which are derived from alkyl-, alkenyl- or polyisobutenylsuccinic anhydride and have amino and/or amido and/or imido and/or hydroxyl groups. It will be appreciated that these reaction products are not obtainable only when substituted succinic anhydride is used, but also when substituted succinic acid or suitable acid derivatives, such as succinyl halides or succinic esters, are used.

In a particularly preferred embodiment, the inventive additized fuel comprises, as further fuel additives, at least one detergent based on a polyisobutenyl-substituted succinimide. Especially of interest are the imides with aliphatic polyamines. Particularly preferred polyamines are ethylenediamine, diethylenetriamine, triethylenetetramine, pentaethylenehexamine and in particular tetraethylenepentamine. The polyisobutenyl radical has a number-average molecular weight Mₙ of preferably from 500 to 5000, more preferably from 500 to 2000 and in particular of about 1000.

Preference is given to using the detergent additives (B) mentioned together with component (A) in combination with at least one carrier oil.

Suitable mineral carrier oils are the fractions obtained in crude oil processing, such as brightstock or base oils having viscosities, for example, from the SN 500-2000 class; but also aromatic hydrocarbons, paraffinic hydrocarbons and alkoxyalkanols. Likewise useful is a fraction which is obtained in the refining of mineral oil and is known as “hydrocrack oil” (vacuum distillate cut having a boiling range of from about 360 to 500° C., obtainable from natural mineral oil which has been catalytically hydrogenated under high pressure and isomerized and also deparaffinized). Likewise suitable are mixtures of abovementioned mineral carrier oils.

Examples of suitable synthetic carrier oils are selected from: polyolefins (poly-alpha-olefins or poly(internal olefin)s), (poly)esters, (poly)alkoxylates, polyethers, aliphatic polyether amines, alkylphenol-started polyethers, alkylphenol-started polyether amines and carboxylic esters of long-chain alkanols.

Examples of suitable polyolefins are olefin polymers having Mₙ=from 400 to 1800, in particular based on polybutene or polyisobutene (hydrogenated or unhydrogenated).

Examples of suitable polyethers or polyetheramines are preferably compounds comprising polyoxy-C₂-C₄-alkylene moieties which are obtainable by reacting C₂-C₆₀-alkanols, C₆-C₃₀-alkanediols, mono- or di-C₂-C₃₀-alkylamines, C₁-C₃₀-alkylcyclo-hexanols or C₁-C₃₀-alkylphenols with from 1 to 30 mol of ethylene oxide and/or propylene oxide and/or butylene oxide per hydroxyl group or amino group, and, in the case of the polyether amines, by subsequent reductive amination with ammonia, monoamines or polyamines. Such products are described in particular in EP-A-310 875, EP-A-356 725, EP-A-700 985 and U.S. Pat. No. 4,877,416. For example, the polyether amines used may be poly-C₂-C₆-alkylene oxide amines or functional derivatives thereof. Typical examples thereof are tridecanol butoxylates or isotridecanol butoxylates, isononylphenol butoxylates and also polyisobutenol butoxylates and propoxylates, and also the corresponding reaction products with ammonia.

Examples of carboxylic esters of long-chain alkanols are in particular esters of mono-, di- or tricarboxylic acids with long-chain alkanols or polyols, as described in particular in DE-A-38 38 918. The mono-, di- or tricarboxylic acids used may be aliphatic or aromatic acids; suitable ester alcohols or polyols are in particular long-chain representatives having, for example, from 6 to 24 carbon atoms. Typical representatives of the esters are adipates, phthalates, isophthalates, terephthalates and trimellitates of isooctanol, isononanol, isodecanol and isotridecanol, for example di-(n- or isotridecyl) phthalate.

Further suitable carrier oil systems are described, for example, in DE-A-38 26 608, DE-A-41 42 241, DE-A-43 09 074, EP-A-0 452 328 and EP-A-0 548 617.

Examples of particularly suitable synthetic carrier oils are alcohol-started polyethers having from about 5 to 35, for example from about 5 to 30, C₃-C₆-alkylene oxide units, for example selected from propylene oxide, n-butylene oxide and isobutylene oxide units, or mixtures thereof. Nonlimiting examples of suitable starter alcohols are long-chain alkanols or phenols substituted by long-chain alkyl in which the long-chain alkyl radical is in particular a straight-chain or branched C₆-C₁₈-alkyl radical. Preferred examples include tridecanol and nonylphenol.

Further suitable synthetic carrier oils are alkoxylated alkylphenols, as described in DE-A-10 102 913.

Preferred carrier oils are synthetic carrier oils, particular preference being given to polyethers.

The detergent additive (B) or a mixture of different such detergent additives is added to the inventive additized fuel in a total amount of preferably from 10 to 2000 ppm by weight, more preferably from 20 to 1000 ppm by weight, even more preferably from 50 to 500 ppm by weight and in particular from 50 to 200 ppm by weight, for example from 70 to 150 ppm by weight.

When a carrier oil is used additionally, it is added to the inventive additized fuel in an amount of preferably from 1 to 1000 ppm by weight, more preferably from 10 to 500 ppm by weight and in particular from 20 to 100 ppm by weight.

Cold flow improvers suitable as further coadditives are, for example, copolymers of ethylene with at least one further unsaturated monomer, for example ethylene-vinyl acetate copolymers.

Corrosion inhibitors suitable as further coadditives are, for example, succinic esters, in particular with polyols, fatty acid derivatives, for example oleic esters, oligomerized fatty acids and substituted ethanolamines.

Demulsifiers suitable as further coadditives are, for example, the alkali metal and alkaline earth metal salts of alkyl-substituted phenol- and naphthalenesulfonates and the alkali metal and alkaline earth metal salts of fatty acid, and also alcohol alkoxylates, e.g. alcohol ethoxylates, phenol alkoxylates, e.g. tert-butylphenol ethoxylates or tert-pentylphenol ethoxylates, fatty acid, alkylphenols, condensation products of ethylene oxide and propylene oxide, e.g. ethylene oxide-propylene oxide block copolymers, polyethyleneimines and polysiloxanes.

Dehazers suitable as further coadditives are, for example, alkoxylated phenol-formaldehyde condensates.

Antifoams suitable as further coadditives are, for example, polyether-modified polysiloxanes.

Cetane number and combustion improvers suitable as further coadditives are, for example, alkyl nitrates other than the inventive branched decyl nitrates, e.g. cyclohexyl nitrate and especially 2-ethylhexyl nitrate, and peroxides, e.g. di-tert-butyl peroxide.

Antioxidants suitable as further coadditives are, for example, substituted phenols, e.g. 2,6-di-tert-butylphenol and 2,6-di-tert-butyl-3-methylphenol, and also phenylenediamines, e.g. N,N′-di-sec-butyl-p-phenylenediamine.

Metal deactivators suitable as further coadditives are, for example, salicylic acid derivatives, e.g. N,N′-disalicylidene-1,2-propanediamine.

Suitable solvents, especially for diesel performance packages, are, for example, nonpolar organic solvents, especially aromatic and aliphatic hydrocarbons, for example toluene, xylenes, “white spirit” and the technical solvent mixtures of the designations Shellsol® (manufacturer: Royal Dutch/Shell Group), Exxol® (manufacturer: ExxonMobil) and Solvent Naphtha. Also useful here, especially in a blend with the nonpolar organic solvents mentioned, are polar organic solvents, in particular alcohols such as 2-ethylhexanol, decanol and isotridecanol.

When the coadditives and/or solvents mentioned are used additionally, they are used in the amounts customary therefor.

The present invention also provides a fuel additive concentrate, for example a diesel performance package, which, based in each case on the total amount of the fuel additive concentrate, comprises
(A) from 0.5 to 80% by weight, especially from 5 to 75% by weight, in particular from 10 to 70% by weight, of at least one inventive branched decyl nitrate or of an inventive mixture of branched decyl nitrates and
(B) from 0.5 to 60% by weight, especially from 3 to 55% by weight, in particular from 5 to 50% by weight, of at least one detergent, especially of at least one detergent based on a polyisobutenyl-substituted succinimide.

In addition, the inventive fuel additive concentrate generally comprises one or more of the further coadditives mentioned above and/or of the solvents mentioned above.

The present invention further provides for the use of the inventive branched decyl nitrates or of the inventive mixture of branched decyl nitrates as combustion improvers and/or cetane number improvers in fuels.

The inventive branched decyl nitrates and the inventive mixture thereof are at least just as effective as the market standard 2-ethylhexyl nitrate as fuel additives with regard to the increase in the cetane number in diesel fuel. In comparison to 2-ethylhexyl nitrate, however, they have a lower volatility, a higher flash point and a higher temperature of autocatalytic decomposition, i.e. better thermal stability. Moreover, they also have a lower percentage nitrogen content (2-ethylhexyl nitrate: 8.0% by weight of N, decyl nitrates: 6.9% by weight of N) and thus ensure a lower content of nitrogen oxides in the exhaust gases. They have a good low-temperature performance, especially a low pour point. Moreover, it has been observed that they reduce the particle emission which increases in the course of engine operation by, among other effects, reducing or preventing the formation of deposits in the intake systems and injection systems (injectors) of the engines. This effect is observed especially in the case of direct-injection diesel engines, especially in common-rail injection systems.

The examples which follow are intended to illustrate the present invention without restricting it.

### EXAMPLE

### Determination of Cetane Numbers with Diesel Fuel

The cetane numbers of the same commercial diesel fuel without cetane number improver (test 1), with 2-ethylhexyl nitrate as a cetane number improver (tests 2 and 4) and with technical-grade 2-n-propylheptyl nitrate which comprised 88% by weight of pure 2-n-propylheptyl nitrate, a total of 11.5% by weight of 2-n-propyl-4-methylhexyl nitrate and 2-n-propyl-5-methylhexyl nitrate and 0.5% by weight of other branched decyl nitrates (of which 0.1% by weight is 2-isopropylheptyl nitrate) (tests 3 and 5), were determined according to the standard EN ISO 5165 in a BASF-MWM engine to DIN 51 773, and corrected for CFR level according to EN 590. The table below shows the results:

| Test | Cetane number improver | Dosage | Result |
|---|---|---|---|
| No. 1 | None | — | 50.3 |
| No. 2 | 2-ethylhexyl nitrate | 500 ppm by wt. | 52.7 |
| No. 3 | Technical-grade 2-n-propylheptyl | 500 ppm by wt. | 52.0 |
| | nitrate | | |
| No. 4 | 2-ethylhexyl nitrate | 1000 ppm by wt. | 53.4 |
| No. 5 | Technical-grade 2-n-propylheptyl | 1000 ppm by wt. | 53.4 |
| | nitrate | | |

At a dosage of 500 ppm by weight of cetane number improver, the value for the technical-grade 2-n-propylheptyl nitrate is in the order of magnitude of the market standard 2-ethylhexyl nitrate; at a dosage of 1000 ppm by weight, the two values are identical.

1. A branched decyl nitrate of the formula I
R¹R²CH—CH₂—O—NO₂ (I)
in which R¹ is an n-propyl or isopropyl radical and R² is a linear or branched alkyl radical having 5 carbon atoms.

2. A process for preparing a branched decyl nitrate according to claim 1, which comprises nitrating the corresponding branched decanol by means of a mixture of nitric acid and sulfuric acid.

3. A mixture of branched decyl nitrates comprising, as components,
from 10 to 99% by weight of 2-n-propylheptyl nitrate,
from 1 to 90% by weight of 2-n-propyl-4-methylhexyl nitrate and/or 2-n-propyl-5-methylhexylnitrate and
from 0 to 50% by weight of other branched decyl nitrates of the formula I according to claim 1,
where the sum of the components adds up to 100% by weight.

4. A process for preparing the mixture of branched decyl nitrates according to claim 3, comprising
(A) hydroformylating butenes to C₅-aldehydes,
(B) dimerizing the C₅-aldehydes by aldol condensation to give the corresponding C₁₀-acrolein derivates,
(C) hydrogenating the C₁₀-acrolein derivates to the corresponding decanols and
(D) nitrating the decanols by means of a mixture of nitric acid and sulfuric acid to give the mixture of branched decyl nitrates.

5. An additized fuel comprising a major proportion of a base fuel, a minor proportion of at least one branched decyl nitrate of the formula I
R¹R²CH—CH₂—O—NO₂ (I)
in which R¹ is an n-propyl or isopropyl radical and R² is a linear or branched alkyl radical having 5 carbon atoms or of a mixture of branched decyl nitrates according to claim 3, and also, optionally, further customary fuel additives in the amounts customary therefor.

6. The additized fuel according to claim 5, comprising from 20 to 2000 ppm by weight of branched decyl nitrates.

7. The additized fuel according to claim 5, comprising a middle distillate fuel as the base fuel.

8. The additized fuel according to claim 5, comprising a gasoline fuel as the base fuel.

9. The additized fuel according to claim 5, comprising, as further fuel additives, at least one detergent based on a polyisobutenyl-substituted succinimide.

10. A fuel additive concentrate comprising, based in each case on the total amount of the fuel additive concentrate,
(A) from 0.5 to 80% by weight of at least one branched decyl nitrate of the formula I
R¹R²CH—CH₂—O—NO₂ (I)
in which R¹ is an n-propyl or isopropyl radical and R² is a linear or branched alkyl radical having 5 carbon atoms or of a mixture of branched decyl nitrates according to claim 3 and
(B) from 0.5 to 60% by weight of at least one detergent.

11. A combustion improver and/or a cetane number improver in a fuel comprising a branched decyl nitrate of the formula I
R¹R²CH—CH₂—O—NO₂ (I)
in which R¹ is an n-propyl or isopropyl radical and R² is a linear or branched alkyl radical having 5 carbon atoms or of a mixture of branched decyl nitrates according to claim 3.
